(19) United States
(12) Patent Application Publication
(10) Pub. No.: US 2009/0041767 A1
(43) Pub. Date: Feb. 12, 2009

(54) PHARMACEUTICAL COMBINATIONS

(76) Inventors: Vanitha Ramakrishnan, Belmont, CA (US); Vinay Bhaskar, San Francisco, CA (US)

Correspondence Address: HOWREY LLP-CA, C/O IP DOCKETING DEPARTMENT, 2941 FAIRVIEW PARK DRIVE, SUITE 200, FALLS CHURCH, VA, 22042-2924, US

(21) Appl. No.: 12/181,201

(22) Filed: Jul. 28, 2008

Related U.S. Application Data
(60) Provisional application No. 60/952,328, filed on Jul. 27, 2007.

(51) Int. Cl. A61K 39/395; A61P 35/04; A61P 35/00

(52) U.S. Cl. 424/133.1

(57) ABSTRACT

Pharmaceutical combinations comprising an α5β1 antagonist in combination with a tyrosine kinase inhibitor. In some embodiments, the α5β1 antagonist is volociximab. In some embodiments, the tyrosine kinase inhibitor is sunitinib or a pharmaceutically acceptable salt thereof. The invention also relates to methods for treating cancer by administering the pharmaceutical combinations to a subject.

### CROSS REFERENCE TO RELATED APPLICATIONS

This application claims priority under 35 U.S.C. § 119(e) of U.S. provisional application No. 60/952,328, filed Jul. 27, 2007, which is hereby incorporated by reference herein.

### FIELD OF THE INVENTION

The invention relates to pharmaceutical combinations comprising an α5β1 integrin antagonist and a tyrosine kinase inhibitor for the prevention or treatment of cancer.

### BACKGROUND OF THE INVENTION

Cancer is a class of diseases or disorders characterized by uncontrolled cell division. Cancer affects people of all ages and is one of the leading causes of death in developed countries. There are many different types of cancer. Once diagnosed, cancer is treated with a combination of surgery, chemotherapy and radiotherapy. However, each of these treatments has numerous undesirable side effects. In addition, because cancer refers to a class of diseases, it is unlikely that there will be a single cure for cancer. Therefore, new treatments for cancer are needed.

The association of α5β1 integrin with tumor angiogenesis is well established. α5β1 is a heterodimeric integrin that is expressed on the surface of endothelial cells and mediates migration toward and adhesion to fibronectin in the extracellular matrix. The binding interaction between α5β1 and fibronectin has been shown to be important for tumor angiogenesis. Angiogenesis within a tumor begins when the release of one or more pro-angiogenic growth factors, such as FGF, VEGF, PDGF, etc., locally activates the endothelial cells. These activated endothelial cells then form new blood vessels by binding, via their α5β1 integrin, to fibronectin in the extracellular matrix. α5β1 antagonists have been shown to inhibit angiogenesis in in vivo tumor models.

Protein kinases are enzymes that catalyze the phosphorylation of hydroxy groups on tyrosine, serine and threonine residues of proteins. There are two types of protein kinases: tyrosine kinases and serine-threonine kinases.

Tyrosine kinases are enzymes that catalyze the phosphorylation of tyrosine residues. There are two types of tyrosine kinases: receptor tyrosine kinases and intracellular tyrosine kinases (non-receptor tyrosine kinases). Tyrosine kinases are involved in cellular signaling pathways and regulate key cell functions, such as cell growth, proliferation, differentiation, anti-apoptotic signaling and neurite outgrowth. Unregulated activation of these enzymes, through mechanisms such as point mutations, etc., can lead to various forms of cancer.

### SUMMARY OF THE INVENTION

Typical embodiments of the invention relate to pharmaceutical combinations comprising an α5β1 antagonist in combination with a tyrosine kinase inhibitor. The pharmaceutical combinations comprise a first amount of an α5β1 antagonist in combination with a second amount of a tyrosine kinase inhibitor, which together comprise a therapeutically effective amount for the prevention or treatment of cancer. In some embodiments, the α5β1 antagonist is volociximab or an antigen binding fragment thereof. In some embodiments, the tyrosine kinase inhibitor is sunitinib or a pharmaceutically acceptable salt thereof. In other embodiments, the tyrosine kinase inhibitor is sorafenib or a pharmaceutically acceptable salt thereof. In further embodiments, the tyrosine kinase inhibitor is bevacizumab or an antigen binding fragment thereof. Other embodiments of the invention relate to methods for preventing or treating cancer by administering the pharmaceutical combinations to a subject.

### BRIEF DESCRIPTION OF THE DRAWINGS

FIGS. 1A and 1B are plots of two separate studies illustrating the effects of the pharmaceutical combination of an α5β1 antagonist, the monoclonal antibody 339.1, and a tyrosine kinase inhibitor, sunitinib (SUTENT®), on tumor volume over time, relative to each agent alone, in a mouse xenograft model of rhabdomyosarcoma (A673).

FIG. 2 is a plot illustrating the effect of the pharmaceutical combination of an α5β1 antagonist, the monoclonal antibody 339.1, and a tyrosine kinase inhibitor, sunitinib (SUTENT®), on tumor volume over time, relative to each agent alone, in a mouse xenograft model of renal cancer (SN12C).

FIG. 3 is a plot illustrating the effect of the pharmaceutical combination of an α5β1 antagonist the monoclonal antibody 339.1, and a tyrosine kinase inhibitor, sunitinib (SUTENT®), on tumor volume, relative to each agent alone, in a mouse xenograft model of renal cancer (786-0).

FIG. 4 is a plot illustrating the effect of the pharmaceutical combination of an α5β1 antagonist, the monoclonal antibody 339.1 (which binds mouse α5β1 with high affinity), its analog, the chimeric antibody volociximab (which binds human α5β1 with high affinity but does not cross-react with mouse α5β1), and a tyrosine kinase inhibitor, sunitinib (SUTENT®), on tumor volume, relative to controls, in a mouse xenograft model of renal cancer (786-0).

FIG. 5 is a plot illustrating the effect of the pharmaceutical combination of an α5β1 antagonist, the monoclonal antibody 339.1 (which binds mouse α5β1 with high affinity), its analog, the chimeric antibody volociximab (which binds human α5β1 with high affinity but does not cross-react with mouse α5β1), and a tyrosine kinase inhibitor, sorafenib (NEXAVAR®) on tumor volume, relative to controls, in a mouse xenograft model of renal cancer (786-0).

FIGS. 6A and 6B are plots of two separate studies illustrating the effects of the pharmaceutical combination of an α5β1 antagonist, the monoclonal antibody 339.1 (which binds mouse α5β1 with high affinity), its analog, the chimeric antibody volociximab (which binds human α5β1 with high affinity but does not cross-react with mouse α5β1), and a tyrosine kinase inhibitor, AVASTIN® on tumor volume, relative to controls, in a mouse xenograft model of renal cancer (A673).

FIGS. 7A and 7B are plots of two separate studies illustrating the effects of the pharmaceutical combination of an α5β1 antagonist, the monoclonal antibody 339.1 (which binds mouse α5β1 with high affinity), its analog, the chimeric antibody volociximab (which binds human α5β1 with high affinity but does not cross-react with mouse α5β1), and a tyrosine kinase inhibitor, AVASTIN® on tumor volume, relative to controls, in a mouse xenograft model of melanoma (LOX).

FIGS. 8A and 8B are plots of two separate studies illustrating the effects of the pharmaceutical combination of an α5β1 antagonist, the monoclonal antibody 339.1 (which binds mouse α5β1 with high affinity), its analog, the chimeric antibody volociximab (which binds human α5β1 with high affinity but does not cross-react with mouse α5β1), and a tyrosine kinase inhibitor, AVASTIN® on tumor volume, relative to controls, in a mouse xenograft model of lung cancer (H460).

### DETAILED DESCRIPTION OF THE INVENTION

All patents and publications, including all sequences disclosed within such patents and publications, referred to herein are expressly incorporated by reference. Unless defined otherwise herein, all technical and scientific terms used herein have the same meaning as commonly understood by one of ordinary skill in the art to which this invention belongs. Any methods and materials similar or equivalent to the various embodiments described herein can be used in the practice or testing of the present invention.

It is intended that every maximum (or minimum) numerical limitation disclosed in this specification includes every lower (or higher) numerical limitation, as if such lower (or higher) numerical limitations were expressly written herein. Moreover, every numerical range disclosed in this specification is intended include every narrower numerical range that falls within such broader numerical range, as if such narrower numerical ranges were all expressly written herein.

As used herein the phrase “at least” when used in combination with a list of values or terms is meant to apply to each value or term in the list. For example, the phrase “at least 85%, 90%, 95% and 99% sequence identity” is used to denote at least 85%, at least 90%, at least 95% and/or at least 99% sequence identity.

As used herein the term “comprising” and its cognates are used in their inclusive sense; that is, equivalent to the term “including” and its corresponding cognates.

Unless otherwise indicated, nucleic acids are written left to right in 5′ to 3′ orientation; amino acid sequences are written left to right in amino to carboxyl orientation, respectively.

The headings provided herein are not limitations of the various aspects or embodiments of the invention that can be had by reference to the specification as a whole. Accordingly, the terms defined below are more fully defined by reference to the specification as a whole.

### Pharmaceutical Combinations

Typical embodiments of the invention relate to pharmaceutical combinations comprising an α5β1 antagonist in combination with a tyrosine kinase inhibitor. The pharmaceutical combinations comprise a first amount of an α5β1 antagonist in combination with a second amount of a tyrosine kinase inhibitor, which together comprise a therapeutically effective amount for the prevention or treatment of cancer. It is contemplated that in some embodiments, the pharmaceutical combinations of the present invention can exhibit a synergistic effect (i. e., additive efficacy) in the treatment of cancers in comparison to treatment with the component pharmaceuticals alone (i.e., not in combination).

The term “pharmaceutical combination,” as used herein, means a combination of two or more medicaments. The medicaments may be administered jointly, such as in a single pill or solution, or separately, such as in two or more separate pills, two or more separate solutions, or one or more solutions and one or more pills. If administered separately, the medicaments may be administered at the same time, sequentially, or within a specified period of time within each other.

The term “therapeutically effective amount”, as used herein, means a first amount of a first medicament in combination with a second amount of a second medicament, and, optionally, additional amounts of additional medicaments, which together will prevent, alleviate, attenuate or treat one or more of the symptoms or complications of cancer. Clinical methods for determining a therapeutically effective amount are well known to those of ordinary skill in the art. For example, a therapeutically effective amount refers to those amounts that, in combination, have the effect of: reducing the size of a tumor; reducing the number of cancer cells; inhibiting tumor growth; inhibiting tumor metastasis; and preventing or relieving, to some extent in a subject, one or more of the symptoms or complications associated with cancer.

The term “subject”, as used herein, means human and non-human mammals.

### α5β1 Antagonists

The pharmaceutical combinations comprise an α5β1 antagonist, a pharmaceutically acceptable salt thereof, or an antigen binding fragment thereof. However, the pharmaceutical combinations may comprise multiple α5β1 antagonists. The term “α5β1 antagonist”, as used herein, means a compound that binds to α5β1 integrin and prevents the integrin from binding to its ligand, fibronectin. Examples of classes of α5β1 antagonists include, but are not limited to, antibodies, peptides and small molecule organic compounds.

In some embodiments, the α5β1 antagonist is an antibody or an antigen binding fragment thereof. As used herein, the term “antibody” means an immunoglobulin molecule that is immunologically reactive with a particular antigen. The term includes monoclonal and polyclonal antibodies, and also genetically engineered forms, such as chimeric antibodies, humanized antibodies, and heteroconjugate antibodies, such as bispecific antibodies, diabodies, triabodies and tetrabodies. Methods for generating monoclonal, polyclonal, chimeric, humanized, and heteroconjugate antibodies useful with present invention are known in the art.

The term “antigen binding fragment” means an antigen binding fragments of an antibody, i.e., a fragment with antigen-binding capability, such as Fab′, F(ab′)₂, Fab, Fv, scFv and rIgG. Methods for generating antigen binding fragments from known antibodies useful with present invention are known in the art.

Examples of antibodies or antigen binding fragments thereof that have α5β1 antagonist activity include, but are not limited to, volociximab (also referred to in some references as “M200”), F200, IIA1, NKI-SAM-1, JBS5, and various humanized antibodies that are disclosed herein. The amino acid sequences and methods for making and using these antibodies and the antigen binding fragments thereof are disclosed in, among other references, U.S. Pat. No. 6,852,318, U.S. Pat. Appl. Publ. No. 2005/0054834 and U.S. Pat. Appl. Publ. No. 2005/0260210, each of which is incorporated herein by reference in its entirety.

In some embodiments, the α5β1 antagonist is volociximab. Volociximab is an anti-α5β1 antibody that is being developed for the treatment of solid tumors. Volociximab is a high-affinity, chimeric IgG4 monoclonal antibody that specifically binds α5β1 integrin. Volociximab is currently in three separate Phase II clinical trials for the treatment of melanoma, renal cell carcinoma and pancreatic cancer.

Volociximab, its preparation, formulation, and use for cancer treatment, are known in the art, and disclosed in, among other references, U.S. Pat. Appl. Publ. No. 2005/0054834 and U.S. Pat. Appl. Publ. No. 2005/0260210. Volociximab comprises a heavy chain comprising the amino acid sequence of SEQ ID NO: 2 and a light chain comprising the amino acid sequence of SEQ ID NO: 4. In some embodiments, the amino acid sequence of the heavy chain is encoded by the nucleic acid sequence of SEQ ID NO: 1 and the amino acid sequence of the light chain is encoded by the nucleic acid sequence of SEQ ID NO: 3.

In some embodiments, the α5β1 antagonist is an antigen binding fragment of volociximab. Such a fragment, its preparation, and its use are disclosed in, among other references, U.S. Pat. Appl. Publ. No. 2005/0054834. Such a fragment comprises a heavy chain variable region comprising the amino acid sequence of SEQ ID NO: 5 and a light chain variable region comprising the amino acid sequence of SEQ ID NO: 6. In some embodiments, the antigen binding fragment is conjugated to a human constant region.

In some embodiments, the α5β1 antagonist is F200, a Fab fragment of volociximab. F200, its preparation, and its use are known in the art. For example, it is disclosed in, among other references, U.S. Pat. Appl. Publ. No. 2005/0054834. Because it is a Fab fragment, the F200 light chain DNA and amino acid sequences are the same as the volociximab light chain DNA and amino acid sequences. F200 comprises a heavy chain comprising the amino acid sequence of SEQ ID NO: 8 and a light chain comprising the amino acid sequence of SEQ ID NO: 4. In some embodiments, the amino acid sequence of the heavy chain is encoded by the nucleic acid sequence of SEQ ID NO: 7.

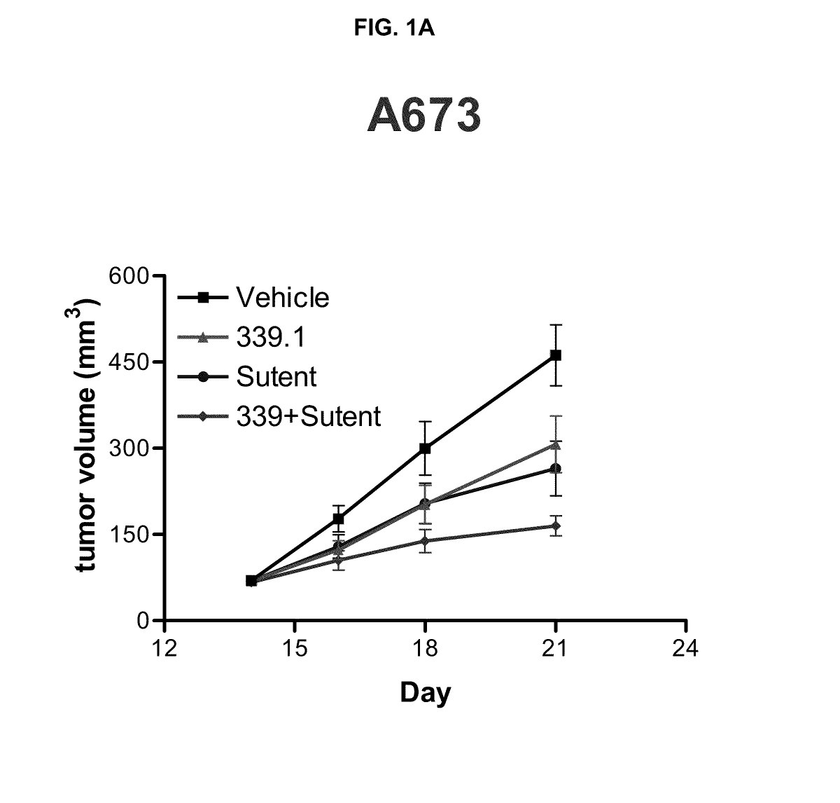

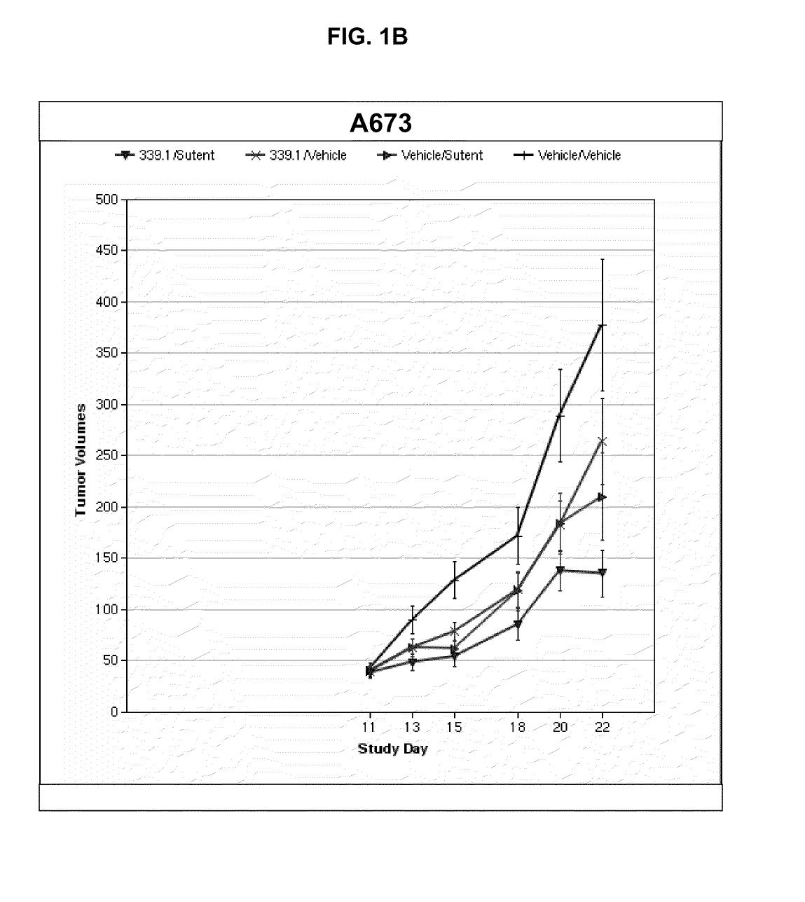

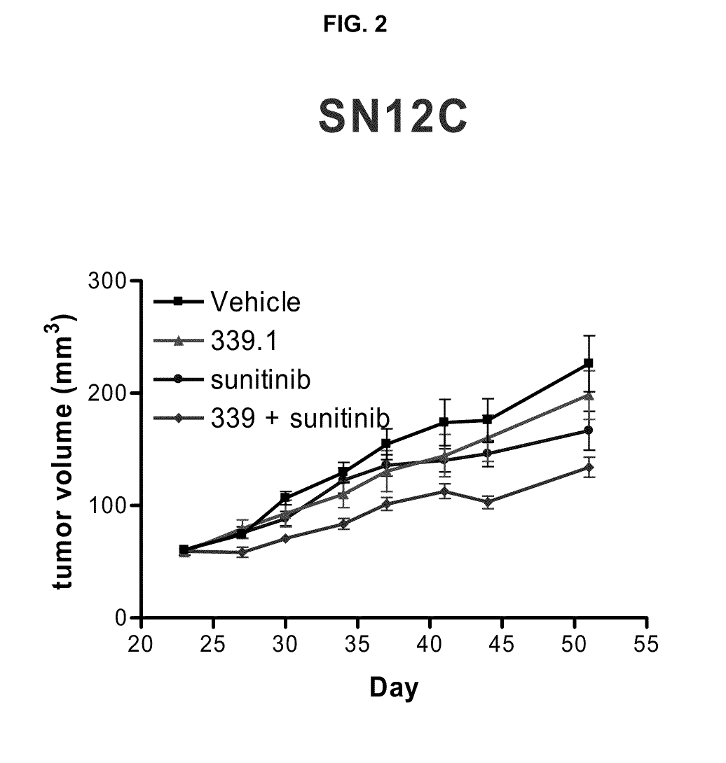

In some embodiments, the α5β1 antagonist is a humanized antibody. Examples of a range of different humanized antibodies that specifically bind human α5β1 integrin are disclosed in U.S. Pat. No. 6,852,318 (see e.g., FIGS. 1 and 2) and described below, wherein the antibodies comprise: a heavy chain variable region comprising the amino acid sequence of SEQ ID NO: 9 and a light chain variable region comprising the amino acid sequence of SEQ ID NO: 10; a heavy chain variable region comprising the amino acid sequence of SEQ ID NO: 11 and a light chain variable region comprising the amino acid sequence of SEQ ID NO: 12; a heavy chain variable region comprising the amino acid sequence of SEQ ID NO: 13 and a light chain variable region comprising the amino acid sequence of SEQ ID NO: 14; a heavy chain variable region comprising the amino acid sequence of SEQ ID NO: 15 and a light chain variable region comprising the amino acid sequence of SEQ ID NO: 16; a heavy chain variable region comprising the amino acid sequence of SEQ ID NO: 17 and a light chain variable region comprising the amino acid sequence of SEQ ID NO: 18; or a heavy chain comprising the amino acid sequence of SEQ ID NO: 19 and a light chain comprising the amino acid sequence of SEQ ID NO: 20.

In some embodiments, the α5β1 antagonist useful in the pharmaceutical combinations of the present invention is an antibody comprising the CDR sequences of an anti-α5β1 antibody selected from volociximab, IIA1, and humanized versions of IIA1. Volociximab, IIA1 and the above-listed humanized anti-α5β1 antibodies share identical CDR sequences, which are depicted in the various amino acid sequences disclosed in U.S. Pat. No. 6,852,318 (see e.g., FIG. 2).

The anti-α5β1 antibody volociximab does not cross-react with murine α5β1 integrin. In other embodiments, the invention provides a monoclonal antibody 339.1 that targets murine α5β1, with binding properties and function blocking characteristics similar to volociximab against human α5β1. The amino acid sequences of the 339.1 heavy chain variable region (SEQ ID NO: 22) and 339.1 light chain variable region (SEQ ID NO: 23) are shown in the attached sequence listing.

In other embodiments, the α5β1 antagonist is a peptide. The term “peptide”, as used herein, includes oligomers and polymers of amino acids or amino acid analogs that are linked together by a peptide bond or an analog of a peptide bond. Thus, a peptide contains two or more amino acids, which can be: either L-amino acids or D-amino acids, chemically modified amino acids, naturally occurring or non-naturally occurring amino acids, or amino acid analogs. α5β1 peptide antagonists can be identified by screening libraries of peptides, can be prepared using well known methods of chemical synthesis, or can be purchased from commercial sources.

Examples of peptides having α5β1 antagonist activity include, but are not limited to, a peptide comprising the amino acid sequence CRRETAWAC (SEQ ID NO: 21) or ATN-161 (Attenuon, San Diego, Calif.). An α5β1 antagonist peptide is disclosed in, among other references, U.S. Pat. No. 6,852,318.

In further embodiments, the α5β1 antagonist is a small molecule organic compound or a pharmaceutically acceptable salt thereof. Examples of small molecule organic compounds having α5β1 antagonist activity include, but are not limited to, a heterocycle having the general chemical structure (S)-2-phenylsulfonylamino-3-{{{8-(2-pyridinyl aminomethyl)-}-1-oxa-2-azaspiro-{4,5}-dec-2-en-yl}carbonylamino}propionic acid. In some embodiments, the compound is SJ749, which has the chemical structure: (S)-2-{(2,4,6-trimethylphenyl) sulfonyl}amino-3-{7-benzyloxycarbonyl-8-(2-pyridinyl aminomethyl)-1-oxa-2,7-diazaspiro-{{4,4}-non-2-en-3-yl}carbonylamino}propionic acid. These compounds, their method of synthesis and their use are disclosed in U.S. Pat. No. 5,760,029, which is incorporated herein by reference in its entirety.

As used herein, the term “pharmaceutically acceptable salt” refers to those salts that retain the pharmacological effectiveness and properties of the parent compound. Such salts include: acid addition salts that are obtained by reaction of the free base of the parent compound with inorganic acids, or salts formed when an acidic proton present in the parent compound is either replaced by a metal ion or coordinates with an organic base. Examples of pharmaceutically acceptable salts are hydrochlorides, sulfates, phosphates, acetates, fumarates, malates, tartarates, carbonates, lactates, maleates, succinates, sodium salts, calcium salts, potassium salts and magnesium salts.

### Tyrosine Kinase Inhibitors

The pharmaceutical combinations comprise a tyrosine kinase inhibitor, a pharmaceutically acceptable salt thereof, or an antigen binding fragment thereof. However, the pharmaceutical combinations may comprise multiple tyrosine kinase inhibitors. The term “tyrosine kinase inhibitor”, as used herein, means a compound that inhibits the phosphorylation of hydroxyl groups on tyrosine residues of proteins, which acts to inhibit cell growth, proliferation, differentiation and apoptotic signaling. Examples of classes of tyrosine kinase inhibitors include, but are not limited to, antibodies, peptides and small molecule organic compounds. Tyrosine kinase inhibition is not limited to mechanism and a tyrosine kinase inhibitor of the invention can act e.g., by antagonizing the function of a tyrosine kinase receptor and/or the ligand for the receptor.

Tyrosine kinase inhibitors are able to antagonize numerous kinds of cellular receptors. For example, some of the receptors that tyrosine kinase inhibitors are able to antagonize, include, but are not limited to, platelet-derived growth factor receptors (PDGFRα and PDGFRβ), vascular endothelial growth factor receptors (VEGFR1, VEGFR2 and VEGFR3), epidermal growth factor receptor (EGFR), stem cell factor receptor (KIT), Fms-like tyrosine kinase-3 (FLT3), colony stimulating factor receptor Type 1 (CSF-1R), Raf kinase, the Src family of kinases, and the glial cell-line derived neurotrophic factor receptor (RET). A tyrosine kinase inhibitor will inhibit one or more of these receptors.

In some embodiments, the tyrosine kinase inhibitor is a small molecule organic compound. Examples of small molecule organic compounds that inhibit tyrosine kinases are: bis-monocylic, bicyclic and heterocyclic aryl compounds, vinyleneazaindole derivatives, 1-cyclopropyl-4-pyridylquinolones, styryl compounds, styryl-substituted pyridyl compounds, quinazoline derivatives, selenaindoles and selenides, tricyclic polyhydroxylic compounds, benzylphosphonic acid compounds, and pyrrole substituted 2-indolinones. These compounds, their preparation and use are disclosed in, among other references, International Appl. Publ. Nos. WO 92/20642, WO 94/14808, WO 94/03427, WO 92/21660, WO 91/15495; U.S. Pat. Nos. 5,330,992, 5,217,999, 5,302,606, 6,573,293, 7,125,905; and European Pat. Appl. Publ. No. EP 0 566 266; each of which is incorporated herein by reference in its entirety.

Additional examples of small molecule organic compounds that inhibit tyrosine kinases are: sorafenib, which is known commercially as NEXAVAR®; dasatinib, which is known commercially as SPRYCEL®; erlotinib, which is known commercially as TARCEVA®; gefitinib, which is known commercially as IRESSA®; imatinib, which is known commercially as GLEEVAC® and GLIVEC®; lapatinib, which is known commercially as TYKERB® and TYCERB®; nilotinib; sunitinib, which is known commercially as SUTENT®; and vandetanib, which is known commercially as ZACTIMA®.

In some embodiments, the tyrosine kinase inhibitor is a pyrrole substituted 2-indolinone. Pyrrole substituted 2-indolinones and their pharmaceutically acceptable salts, their preparation, and their use are known in the art. For example, they are disclosed in, among other references, U.S. Pat. Nos. 6,573,293 and 7,125,905, each of which is hereby incorporated by reference herein. Examples of pharmaceutically acceptable salts of pyrrole substituted 2-indolinones include, but are not limited to, hydrochlorides, sulfates, phosphates, acetates, fumarates, malates, tartarates, carbonates, lactates, maleates, succinates, sodium salts, calcium salts, potassium salts and magnesium salts.

In some embodiments, the pyrrole substituted 2-indolinone is sunitinib. A preferred pharmaceutically acceptable salt of sunitinib is sunitinib malate. Sunitinib is a multi-kinase inhbitor targeting several receptor tyrosine kinases. Sunitinib malate, which is known commercially as SUTENT®, is described chemically as butanedioic acid, hydroxy-, (2S)-, compound with N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide (1:1). Sunitinib malate is a small molecule organic compound that inhibits multiple receptor tyrosine kinases, some of which are implicated in tumor growth, pathologic angiogenesis, and metastatic progression of cancer. For example, sunitinib is an inhibitor of platelet-derived growth factor receptors (PDGFRα and PDGFRβ), vascular endothelial growth factor receptors (VEGFR1, VEGFR2 and VEGFR3), stem cell factor receptor (KIT), Fms-like tyrosine kinase-3 (FLT3), colony stimulating factor receptor Type 1 (CSF-1R), and the glial cell-line derived neurotrophic factor receptor (RET). SUTENT®, which is sold by Pfizer (New York, N.Y.), is indicated for the treatment of both advanced renal cell carcinoma and gastrointestinal stromal tumor after disease progression on or intolerance to imatinib mesylate.

In other embodiments, the tyrosine kinase inhibitor is an aryl urea compound. Aryl urea compounds and their pharmaceutically acceptable salts, their preparation, and their use are known in the art. For example, they are disclosed in, among other references, U.S. Pat. Appl. Publ. Nos. 2003/0216446, 2003/0232765 and 2004/0023961, each of which is hereby incorporated by reference herein. Examples of pharmaceutically acceptable salts of aryl urea compounds include, but are not limited to, hydrochlorides, sulfates, phosphates, acetates, fumarates, malates, tartarates, carbonates, lactates, maleates, succinates, sodium salts, calcium salts, potassium salts and magnesium salts.

In some embodiments, the aryl urea compound is sorafenib. A preferred pharmaceutically acceptable salt of sorafenib is sorafenib tosylate. Sorafenib tosylate, which is known commercially as NEXAVAR®, has the chemical name 4-(4-{3-[4-Chloro-3-(trifluoromethyl)phenyl]ureido}phenoxy)N²-methylpyridine-2-carboxamide 4-methylbenzenesulfonate. Sorafenib is a small molecule organic compound that inhibits multiple receptor tyrosine kinases, some of which are implicated in tumor growth, pathologic angiogenesis, and metastatic progression of cancer. For example, sorafenib is an inhibitor of CRAF, BRAF, KIT, FLT-3, VEGFR-2, VEGFR-3 and PDGFR-β. NEXAVAR®, which is sold by Onyx Pharmaceuticals (Emeryville, Calif.), and is indicated for the treatment of renal cell carcinoma.

In further embodiments, the tyrosine kinase inhibitor is an antibody or an antigen binding fragment thereof that antagonizes the function of a tyrosine kinase. Function blocking can occur by antibody binding to the tyrosine kinase receptor and/or to the ligand (e.g., growth factor) that binds the receptor. In some embodiments, the tyrosine kinase inhibitor is bevacizumab. Bevacizumab is a recombinant humanized monoclonal IgG1 antibody that binds to and inhibits the biologic activity of human vascular endothelial growth factor (VEGF). Bevacizumab, its preparation, and its use are known in the art. For example, it is disclosed in, among other references, Presta, et al., “Humanization of an Anti-Vascular Endothelial Growth Factor Monoclonal Antibody for the Therapy of Solid Tumors and Other Disorders”, Cancer Research, vol. 57, pp. 4593-4599 (1997). Bevacizumab, which is known commercially as AVASTIN®, is sold by Genentech (South San Francisco, Calif.). Bevacizumab is indicated for the treatment of metastatic carcinoma of the colon or rectum; and metastatic non-squamous, non-small cell lung cancer.

### Formulation & Administration

Each of the medicaments in the pharmaceutical combinations may be administered in an isolated and purified form, and directly contacted with cancer cells or tumors. Methods of making and purifying α5β1 antagonists and tyrosine kinase inhibitors are disclosed in the references cited herein. Purity and homogeneity may be determined using standard analytical chemistry techniques, such as polyacrylamide gel electrophoresis and high performance liquid chromatography. A compound that is the predominant species present in a preparation is considered to be substantially purified. For example, a compound that exhibits essentially one band in an electrophoretic gel is substantially purified. In some embodiments, each of the medicaments used in the pharmaceutical combinations of the invention is at least 85% pure, at least 95% pure, or even at least 99% pure.

Alternatively, the medicaments of the pharmaceutical combinations may be formulated into one or more pharmaceutical compositions before administration. As used herein, the term “pharmaceutical composition” means a medicament that is formulated in a pharmaceutically acceptable carrier or excipient. Therefore, the medicaments of the pharmaceutical combinations may be formulated into: separate pharmaceutical compositions, or into one or more pharmaceutical compositions. Such a pharmaceutical composition will commonly comprise a medicament that is formulated in a pharmaceutically acceptable carrier or pharmaceutically acceptable excipient. Proper formulation is dependent upon the route of administration chosen. Techniques for formulation and administration of drugs (medicaments) may be found in “Remington's Pharmacological Sciences,” Mack Publishing Co., Easton, Pa.

As used herein, the terms “pharmaceutically acceptable carrier” and “pharmaceutically acceptable excipient” refer to a carrier, excipient or diluent that facilitates administration of a medicament, does not cause significant irritation to a subject and does not abrogate the pharmacological activity and properties of the administered medicament, and is non-toxic to the cell or subject being exposed thereto at the dosages and concentrations employed. Examples of pharmaceutically acceptable carriers and excipients include, but are not limited to, buffers, cosolvents, tonicity agents, pH adjusting agents, antioxidants, sugars, gelatin, gum, pigments, binders, lubricants, fillers, disintegrants, preservatives, flavorings, thickeners, coloring agents and emulsifiers.

A pharmaceutical composition comprising an α5β1 antagonist or a tyrosine kinase inhibitor may be administered to a subject in a variety of ways, including, but not limited to, orally, subcutaneously, intravenously, intravitreally, intranasally, topically, transdermally, transmucosally, intraperitoneally, intramuscularly, intrapulmonary, vaginally, rectally, intraocularly, intraventricularly, or intrathecally. In some embodiments, the composition is administered intravenously or orally.

Pharmaceutical compositions may be manufactured by processes that are well known in the art, for example, by means of conventional mixing, dissolving, granulating, dragee-making, levigating, emulsifying, encapsulating, entrapping or lyophilizing processes. Pharmaceutical compositions may be manufactured as pills, solutions, tablets, capsules, liquids, gels, suspensions, dragee cores, aerosol sprays, suppositories, etc., which will depend upon the route of administration chosen.

For injection, a medicament may be formulated in an aqueous solution. In some embodiments, the solution is 5% dextrose in water, 0.9% saline, or a physiologically compatible buffer, such as Hanks' solution or Ringer's solution. Additionally, suspensions of the active compounds (medicaments) may be prepared in a lipophilic vehicle or a liposome. Suitable lipophilic vehicles include fatty oils, such as sesame oil; synthetic fatty acid esters, such as ethyl oleate and triglycerides; or materials such as liposomes. Aqueous injection suspensions may also contain substances that increase the viscosity of the suspension, such as sodium carboxymethyl cellulose, sorbitol, or dextran. Optionally, the suspension may also contain suitable stabilizers and/or agents that increase the solubility of the compounds in order to allow for the preparation of highly concentrated solutions. Alternatively, the active ingredient (medicament) may be in powder form for constitution with a suitable vehicle, such as sterile, pyrogen-free water, 5% dextrose in water or 0.9% saline, before use.

A medicament may be formulated for parenteral administration, for example, by bolus injection or continuous infusion. Formulations for injection may be presented in unit dosage form, such as ampoules, vials, multi-dose containers, intravenous bags or bottles.

For transmucosal administration, penetrants appropriate for the barrier to be permeated are used in the formulation. Such penetrants are generally known in the art.

For oral administration, a medicament may be formulated by combining it with a pharmaceutically acceptable carrier. Such a carrier enables the medicament to be formulated as a tablet, pill, lozenge, dragee, capsule, liquid, gel, syrup, slurry, suspension and the like, for oral ingestion. Pharmaceutical compositions for oral use can be made using a solid excipient, optionally grinding the resulting mixture, and processing the mixture of granules, after adding other suitable auxiliaries if desired, to obtain tablets or dragee cores. Useful excipients include fillers, such as sugars, including lactose, sucrose, mannitol, or sorbitol; cellulose preparations, such as maize starch, wheat starch, rice starch and potato starch; and other materials, such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar or alginic acid. A salt such as sodium alginate may also be used.

A medicament may be formulated as a dragee core. For this purpose, concentrated sugar solutions may be used that may, optionally, contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablet or dragee coating for identification or to characterize different combinations of active compound doses.

A medicament may also be formulated as a push-fit capsule made of gelatin, as well as a soft, sealed capsule made of gelatin and a plasticizer, such as glycerol or sorbitol. The push-fit capsule may contain a medicament in admixture with a filler, such as lactose; a binder, such as starch; and/or a lubricant, such as talc or magnesium stearate; and, optionally, stabilizers. In soft capsules, a medicament may be dissolved or suspended in suitable liquids, such as fatty oils, liquid paraffin, or liquid polyethylene glycols. Alternatively, a medicament may be formulated as a hard gelatin capsule.

For administration by inhalation, a medicament may be delivered in the form of an aerosol spray using a pressurized pack or a nebulizer and a suitable propellant, such as dichlorodifluoromethane, trichlorofluoromethane, dichlorotetra-fluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of a medicament and a suitable powder base, such as lactose or starch.

A medicament may also be formulated in a rectal composition, such as suppository or retention enema, using, for example, conventional suppository bases, such as cocoa butter or other glycerides.

In addition, a medicament may be formulated as a depot preparation. Such a long acting composition may be administered by implantation, for example, subcutaneously, or by intramuscular injection. A medicament may be formulated for this route of administration with a suitable polymeric or hydrophobic material, with ion exchange resins, or as a sparingly soluble salt.

Additionally, a medicament may be delivered using a sustained-release system, such as a semipermeable matrix of solid hydrophobic polymers containing the medicament. Various sustained-release materials are well known by those skilled in the art. Sustained-release capsules may, depending on their chemical nature, release the medicament over a period of a few days up to 100 days. Depending upon the chemical nature and stability of the medicament, additional strategies for stabilization may be employed.

In some embodiments, the α5β1 antagonists are anti-α5β1 antibodies, including antigen binding fragments thereof, which are formulated as a pharmaceutical composition comprising a solution of 1.0 mg/mL to 15.0 mg/mL α5β1 antagonist, 22 mM to 28 mM citrate, 135 mM to 165 mM sodium chloride, 0.04%-0.06% polysorbate (TWEEN®) 80, at a pH of 5.5 to 7.5. In some embodiments, the pH range of the liquid formulation is between pH 6.0 and pH 7.0, between pH 6.3 and pH 6.7, between pH 6.4 and 6.6, or about pH 6.5. Preferably, the composition is stable and isotonic. In a particularly preferred embodiment, the pharmaceutical composition comprises a solution of 10.0 mg/mL α5β1 antagonist, 25 mM citrate, 150 mM sodium chloride, 0.05% polysorbate (TWEEN®) 80, at a pH of 6.5. Preferably, the composition is refrigerated at 2-8° C.

Exemplary formulations, dosages, methods of administering, and other therapeutic protocols for anti-α5β1 antibodies useful in the pharmaceutical combinations of the present invention are disclosed in U.S. Pat. Appl. Publ. No. 2005/0260210, which is hereby incorporated by reference herein.

In some embodiments, sunitinib, including its pharmaceutically acceptable salts, are formulated as a pharmaceutical composition comprising a capsule containing sunitinib malate equivalent to 12.5 mg, 25 mg or 50 mg of sunitinib, mannitol, croscarmellose sodium, povidone and magnesium stearate.

In some embodiments, sorafenib, including its pharmaceutically acceptable salts, are formulated as a pharmaceutical composition comprising a tablet containing sorafenib tosylate equivalent to 200 mg sorafenib, croscarmellose sodium, microcrystalline cellulose, hypromellose, sodium lauryl sulphate, magnesium stearate, polyethylene glycol, titanium dioxide and ferric oxide red.

In some embodiments, bevacizumab, including antigen binding fragments thereof, are formulated as a pharmaceutical composition comprising either 100 mg or 400 mg of bevacizumab in 4 ml or 16 ml single use vials, respectively. The 100 mg, 4 ml vial, is formulated in 240 mg α,α-trehalose dihydrate, 23.2 mg sodium phosphate (monobasic, monohydrate), 4.8 mg sodium phosphate (dibasic, anhydrous), 1.6 mg polysorbate 20, and water. The 400 mg, 16 ml vial, is formulated in 960 mg α,α-trehalose dihydrate, 92.8 mg sodium phosphate (monobasic, monohydrate), 19.2 mg sodium phosphate (dibasic, anhydrous), 6.4 mg polysorbate 20, and water. Preferably, each of the compositions is refrigerated at 2-8° C. In addition, it is preferred that the compositions are protected from light. It is also preferred that the necessary amount of bevacizumab is withdrawn from the vial and diluted in 100 ml of 0.9% saline before administration to a subject.

The medicaments may be administered in a variety of unit dosage forms depending upon the method of administration. The exact dosage to be used and frequency of administration in a particular embodiment of the invention will depend upon the purpose of the treatment, and may be ascertained by one of skill in the art using well-known techniques. As is known in the art, adjustments for systemic versus localized delivery, age, body weight, general health, sex, diet, time of administration, drug interaction and the severity of the condition may be necessary.

Dosage amount and interval may be adjusted individually to provide plasma levels of the medicaments that are sufficient to maintain a pharmacological effect. These plasma levels are referred to as minimal effective concentrations (MECs). The MEC will vary for each medicament, but can be estimated from in vitro data. Dosages necessary to achieve the MEC will depend upon individual characteristics and route of administration. HPLC assays or bioassays can be used to determine plasma concentrations. Dosage intervals can also be determined using MEC value. Medicaments should be administered using a regimen that maintains plasma levels above the MEC for 10-90% of the time, preferably between 30-90% and most preferably between 50-90%.

In some embodiments, a medicament is administered to a subject based on the weight of the medicament (such as in mg) per patient body weight (such as in kg) (i.e., “mg/kg”). In other embodiments, a medicament is administered to a subject based on the weight of the medicament (such as in mg). In further embodiments, a medicament is administered to a subject based on the amount of drug (such as in mM) per amount of blood volume (such as in ml).

Generally, an effective dose of an α5β1 antagonist ranges from 1-15 mg/kg. Preferably, the dose is 10-15 mg/kg. Preferably, α5β1 antibody antagonists, or antigen binding fragments thereof, are administered to a subject based on the weight of antibody (in mg) per patient body weight (in kg) and comprise 0.1-15 mg/kg of α5β1 antagonist antibody, such as volociximab, or an antigen binding fragment thereof, such as F200. For example, suitable concentrations of antibody or an antigen binding fragment thereof, include, but are not limited to: 0.1-15 mg/kg, 0.5 mg/kg, 1.0 mg/kg, 1.0-5.0 mg/kg, 2.5 mg/kg, 5 mg/kg, 5-10 mg/kg, 7.5 mg/kg, 10 mg/kg, 10-15 mg/kg, 12.5 mg/kg or 15 mg/kg. Preferably, the concentration is either 10 or 15 mg/kg.

Exemplary dosages and regimens for anti-α5β1 antibodies useful in the pharmaceutical combinations of the present invention are disclosed in U.S. Pat. Appl. Publ. No. 2005/0260210, which is hereby incorporated by reference herein.

Preferably, the α5β1 peptide and small molecule organic compound antagonists are administered in concentrations of 1-100 picomoles/ml of blood volume. Preferably, the dose is administered to the subject as an intravenous infusion over 1 hour. Additional doses may be administered over an extended time period, such that a steady state serum concentration is established in the subject. For example, an infusion of 10 mg/kg may be administered once a week over the course of a year.

Generally, an effective dose of a tyrosine kinase inhibitor ranges from 1-1000 mg. Preferably, the dose is 1-600 mg.

Preferably, sunitinib is administered to a subject based on the weight of the drug such that a pharmaceutical composition comprises 0.1-100 mg sunitinib or a pharmaceutically acceptable salt thereof. For example, suitable concentrations of sunitinib or a pharmaceutically acceptable salt thereof, include, but are not limited to: 0.1-100 mg, 12.5 mg, 0.1-25 mg, 25 mg, 25-50 mg, 37.5 mg, 50 mg, 50-75 mg, 62.5 mg, 75 mg, 75-100 mg, 87.5 mg and 100 mg.

Sunitinib malate is known commercially as SUTENT®. SUTENT® capsules are supplied as printed hard shell capsules containing sunitinib malate equivalent to 12.5, 25 or 50 mg of sunitinib. The recommended dose of SUTENT® for the treatment of gastrointestinal stromal tumor and renal cell carcinoma is one 50 mg oral dose taken once daily, on a schedule of 4 weeks on treatment followed by two weeks off. Dose increase or reduction of 12.5 mg increments is recommended based on individual safety and tolerability.

Preferably, sorafenib is administered to a subject based on the weight of the drug such that a pharmaceutical composition comprises 1-800 mg sorafenib or a pharmaceutically acceptable salt thereof. For example, suitable concentrations of sorafenib or a pharmaceutically acceptable salt thereof, include, but are not limited to, 200 mg, 400 mg, 600 mg and 800 mg.

Sorafenib tosylate is known commercially as NEXAVAR®. NEXAVAR® tablets are supplied as red, round, film-coated tablets that contain sorafenib tosylate equivalent to 200 mg sorafenib. The recommended dose of NEXAVAR® for the treatment of renal cell carcinoma is 400 mg (two, 200 mg tablets) taken twice daily.

Preferably, bevacizumab, or an antigen binding fragment thereof, is administered to a subject based on the weight of antibody (in mg) per patient body weight (in kg) and comprises 0.1-20 mg/kg of bevacizumab, or an antigen binding fragment thereof. For example, suitable concentrations of antibody or an antigen binding fragment thereof, include, but are not limited to: 0.1-20 mg/kg, 1.0-5.0 mg/kg, 2.5 mg/kg, 5 mg/kg, 5-10 mg/kg, 7.5 mg/kg, 10 mg/kg, 10-15 mg/kg, 12.5 mg/kg, 15 mg/kg, 17.5 mg/kg or 20 mg/kg.

Where the individual medicaments of a pharmaceutical combination are administered separately, the number of doses of each medicament given per day may not necessarily be the same. For example, one medicament may have a greater duration of activity and will need to be administered less frequently.

### Kits

Since some embodiments of the invention relate to the prevention or treatment of cancer by administering a combination of medicaments wherein each of the medicaments may be administered separately, the invention also relates to combining the separate medicaments or pharmaceutical compositions in kit form. That is, a kit is contemplated wherein two or more separate units are combined: one or more α5β1 antagonist pharmaceutical compositions and one or more tyrosine kinase inhibitor pharmaceutical compositions. The kit will preferably include directions for the administration of the separate components. Such a kit form is particularly advantageous when the separate components must be administered in different dosage forms, for example, oral and parenteral, or are administered at different dosage intervals.

### Indications

The pharmaceutical combinations provide a therapeutic approach for the treatment of many kinds of solid tumors, including but not limited to, renal cell carcinoma, melanoma, pancreatic cancer, gastrointestinal stromal tumor, bladder cancer, breast cancer, colon cancer, fibrosarcoma, lung cancer, metastatic melanoma, prostate cancer, ovarian cancer and spleen cancer. In some embodiments, the present invention provides pharmaceutical combinations for treatment of any cancer that expresses α5β1 integrin on the surface of its tumor cells. Tumor cells that express α5β1 integrin on their surface are known in the art and disclosed in e.g., U.S. Pat. Appl. Publ. No. 2005/0260210, which is hereby incorporated by reference herein.

The manner and method of carrying out the present invention may be more fully understood by those of skill in the art by reference to the following examples, which examples are not intended in any manner to limit the scope of the present invention or of the claims directed thereto.

### EXAMPLES

### Example 1

This example illustrates a study of the effect of the tyrosine kinase inhibitor, SUTENT® in combination with the α5β1 integrin antagonist, 339.1 antibody, which is a volociximab analog. The study was performed using three different pre-clinical mouse xenograft tumor models of cancer: rhabdomysosarcoma (A673), renal cancer (SN-12C), and renal cancer (786-0).

The surrogate antibody, 339.1 was used, rather than volociximab, in these mouse xenograft experiments because volociximab does not cross-react with murine α5β1 integrin. The 339.1 antibody targets murine α5β1, with properties similar to volociximab, which targets human α5β1. The amino acid sequences of the 339.1 heavy chain variable region (SEQ ID NO: 22) and 339.1 light chain variable region (SEQ ID NO: 23) are shown in the attached sequence listing. Thus, it was contemplated that the use of the mouse analog of volociximab would provide a better measure of efficacy because it could target angiogenic effects on xenograft tumor growth due to murine α5β1 integrin.

Mice bearing established A673, SN-12C, or 786-0 xenograft tumors were treated with vehicle, 339.1, SUTENT®, or a combination of 339.1 and a sub-maximal dose of SUTENT®. The 339.1 antibody was administered intraperitoneally twice weekly at 10 mg/kg, and SUTENT® was dosed orally on a daily basis at 20 mg/kg.

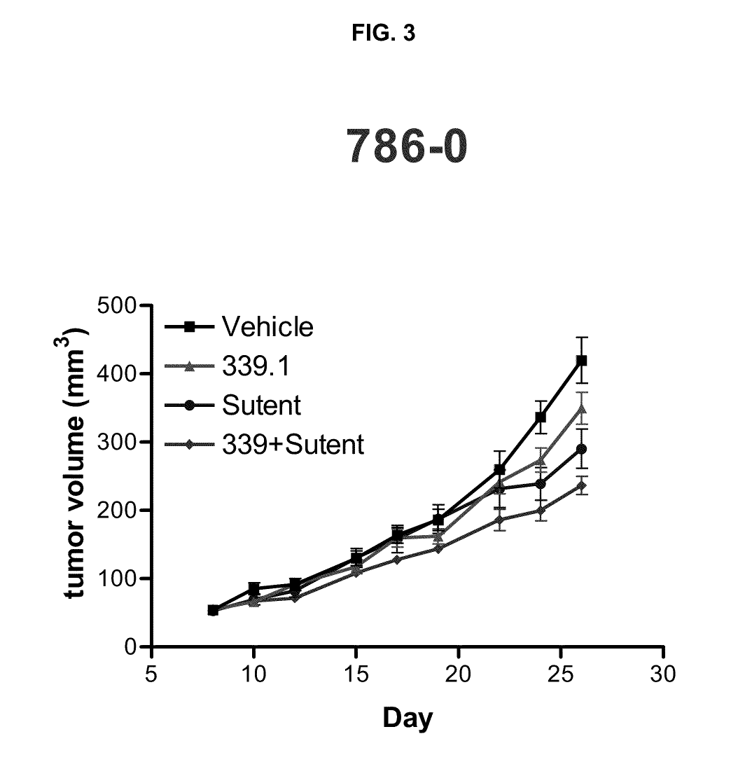

The xenograft tumor growth versus treatment day plots for the studies with tumor models A673, SN-12C and 786-0 are shown in FIGS. 1, 2 and 3, respectively. The results suggest a trend for greater tumor reduction when an anti-α5β1 antagonist is used in combination with the tyrosine kinase inhibitor, SUTENT®.

### Example 2

This example illustrates a study of the combinatorial effects of SUTENT®, volociximab and the voloxicimab analog, 339.1, using a pre-clinical model of cancer. Specifically, the studies were performed using a mouse xenograft tumor model of renal cancer (786-0).

Volociximab was used in this study in addition to the volociximab surrogate antibody, 339.1 in order to model the full anti-tumor effect of treatment of a human cancer patient using a pharmaceutical composition comprising volociximab. The 339.1 antibody is able to target the murine vasculature that develops to support the tumor xenograft model while volociximab is able to target the human α5β1 integrin in the human 786.0 tumor cells. Consequently, it is expected that the combination of volociximab and 339.1 in the xenograft model will more closely model the results of clinical study of volociximab in humans by targeting both the tumor and the vasculature.

In these experiments, mice bearing established xenograft tumors were treated with vehicle and vehicle, vehicle and SUTENT®, 339.1+volociximab and vehicle, or 339.1+volociximab and SUTENT®. The 339.1 antibody and volociximab were administered intraperitoneally twice weekly at 10 mg/kg, except for the first dose of 339.1 at 25 mg/kg, and SUTENT® was dosed orally on a daily basis at 20 mg/kg.

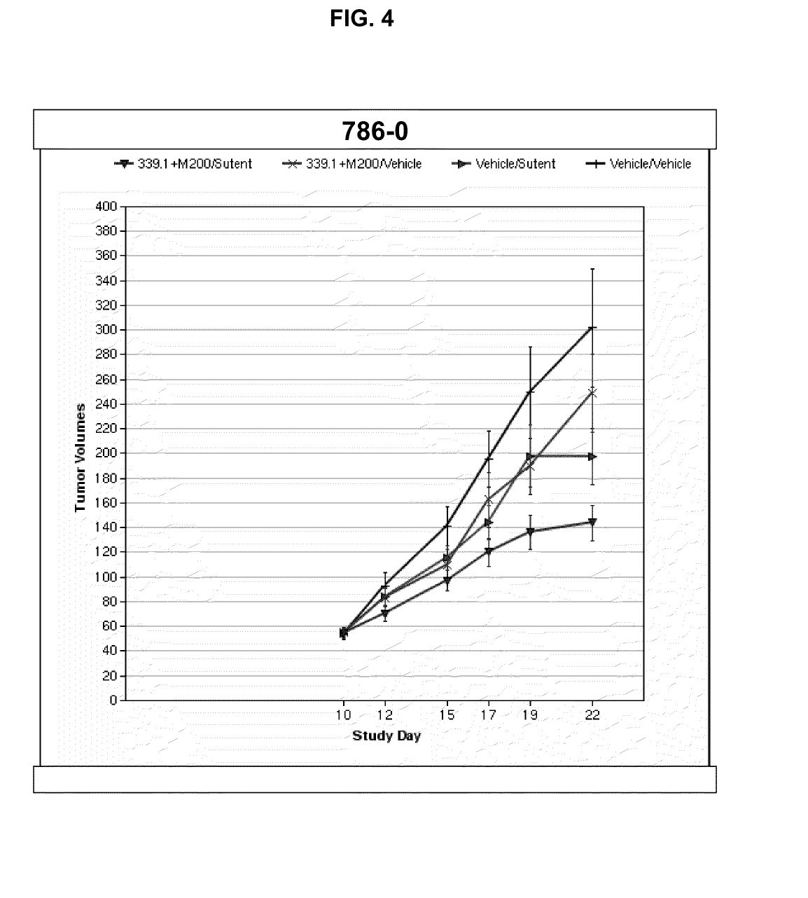

The results of the study are illustrated by the data plotted in FIG. 4. Statistically significant effects were observed with the combination of SUTENT®, 339.1 and volociximab, relative to controls, in the 786-0 model of renal cancer.

These results support the use of a pharmaceutical combination of volociximab and SUTENT® (sunitinib) in patients with cancer, especially renal cell carcinoma.

### Example 3

This Example illustrates a study of the combinatorial effects of the pharmaceutical combination of NEXAVAR®, volociximab and the voloxicimab analog, 339.1 using the mouse xenograft tumor model of renal cancer (786-0).

As explained above in Example 2, voliciximab was used in addition to the surrogate antibody, 339.1, because the 339.1 antibody is able to target the murine vasculature that develops to support the tumor xenograft model while volociximab is able to target the human α5β1 integrin in the human 786.0 tumor cells. Consequently, it is expected that the combination of volociximab and 339.1 in the xenograft model will more closely model the results of clinical study of volociximab in humans by targeting both the tumor and the vasculature.

In these experiments, mice bearing established xenograft tumors were treated with vehicle and vehicle, vehicle and NEXAVAR®, 339.1+volociximab and vehicle, or 339.1+volociximab and NEXAVAR®. The 339.1 antibody and volociximab were administered intraperitoneally twice weekly at 10 mg/kg, except for the first dose of 339.1 at 25 mg/kg, and NEXAVAR® was dosed orally on a daily basis at 12.5 mg/kg.

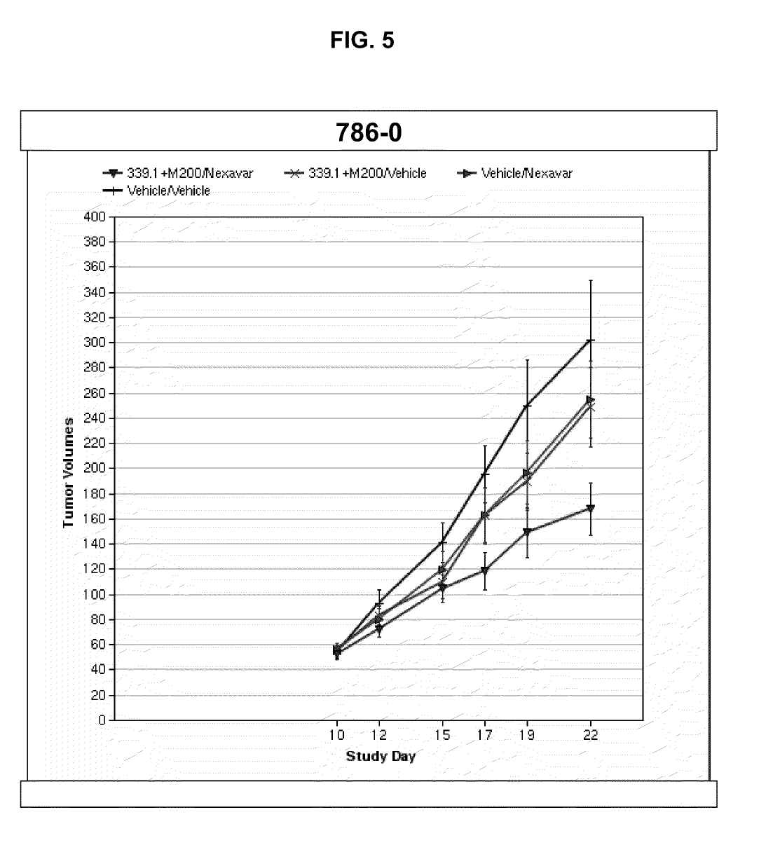

The results of the experiments are illustrated by the plots of tumor volume versus study day shown in FIG. 5. Statistically significant effects, relative to controls, were observed in the 786-0 model of renal cancer with the pharmaceutical combination of NEXAVAR®, 339.1 and volociximab.

These results support the use of a pharmaceutical combination of volociximab and NEXAVAR® (sorafenib) in patients with cancer, especially renal cell carcinoma.

### Example 4

This Example illustrates a study of the combinatorial effects of AVASTIN®, volociximab and the voloxicimab analog, 339.1, using three different pre-clinical models of cancer. Specifically, the studies were performed using mouse xenograft tumor models of rhabdomyosarcoma (A673), melanoma (LOX) and lung cancer (H460). AVASTIN® (bevacizumab) is a marketed cancer therapeutic that acts to inhibit the tyrosine kinase VEGF receptor by binding its VEGF growth factor ligand.

As explained above in Example 2, voliciximab was used in addition to the surrogate antibody, 339.1, because the 339.1 antibody is able to target the murine vasculature that develops to support the tumor xenograft model while volociximab is able to target the human α5β1 integrin in the human 786.0 tumor cells. Consequently, it is expected that the combination of volociximab and 339.1 in the xenograft model will more closely model the results of clinical study of volociximab in humans by targeting both the tumor and the vasculature.

In these experiments, mice bearing established xenograft tumors were treated with vehicle and vehicle, vehicle and AVASTIN®, 339.1+volociximab, or 339.1+volociximab and AVASTIN®. In studies with A673 xenografts, the 339.1 antibody and volociximab were administered intraperitoneally twice weekly at 10 mg/kg, and AVASTIN® was administered twice weekly at 0.5 mg/kg. For studies with the LOX and H460 xenografts, the 339.1 antibody and volociximab were administered intraperitoneally twice weekly at 10 mg/kg, with the exception of some studies where a first dose of 339.1 at 25 mg/kg was used, and AVASTIN® was administered twice weekly at 10 mg/kg.

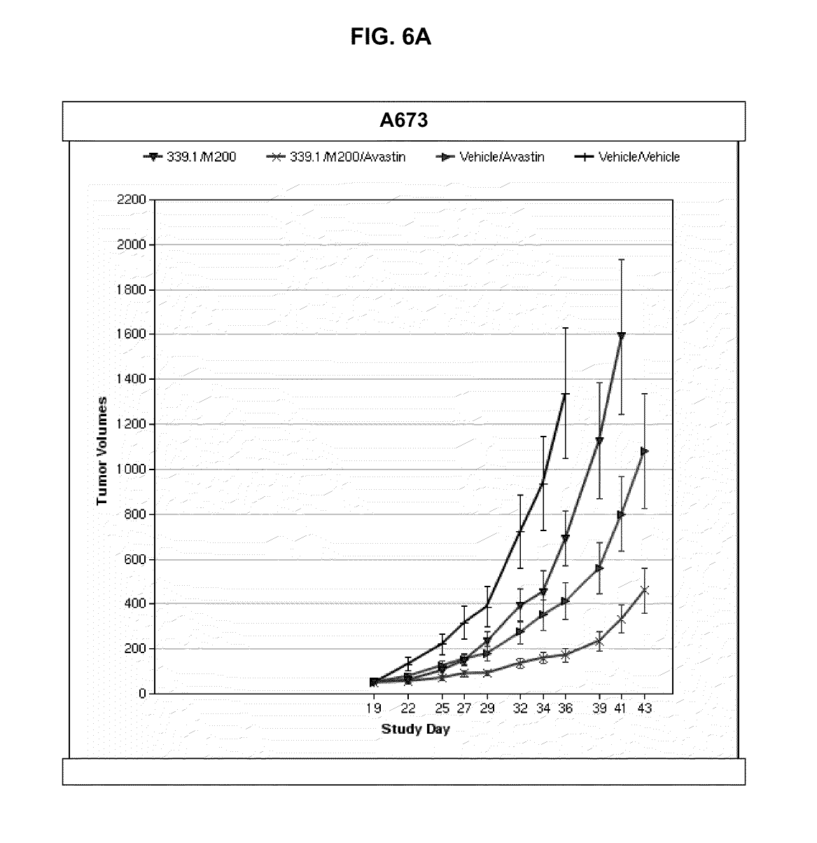

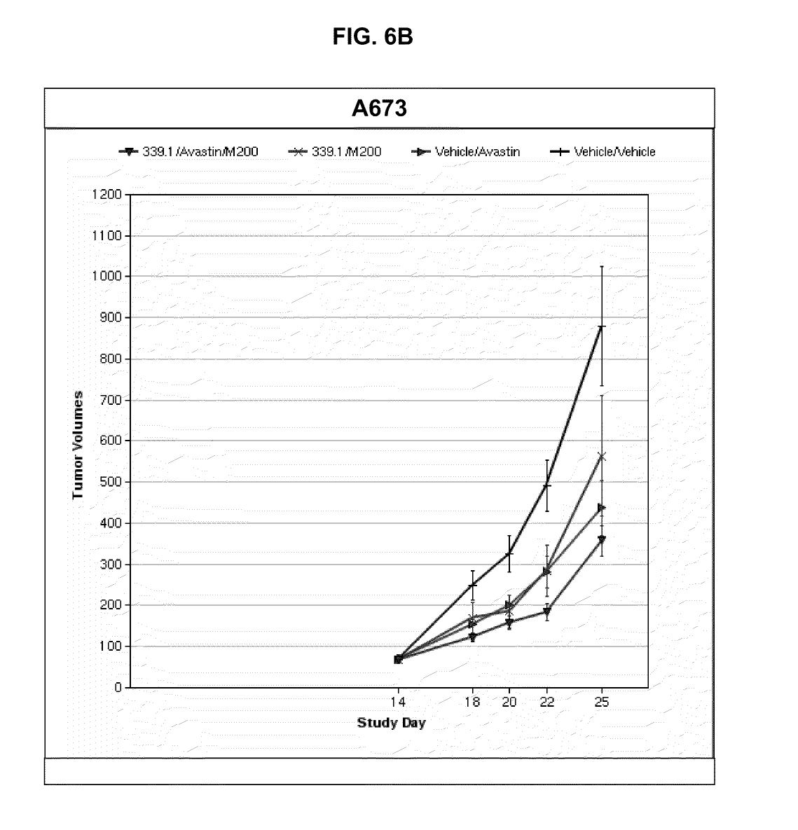

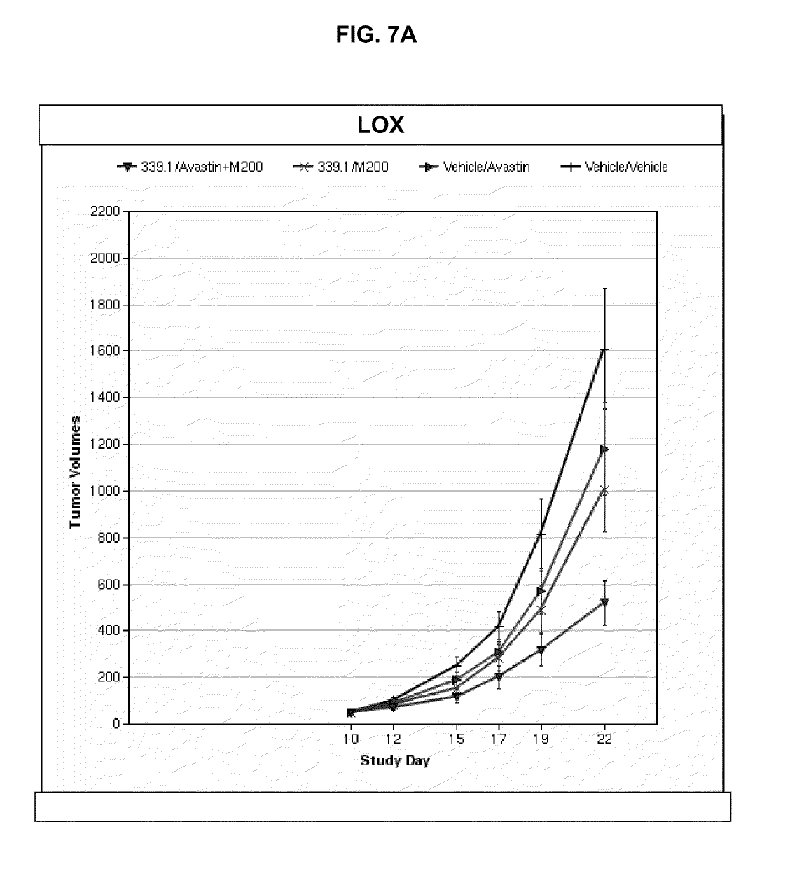

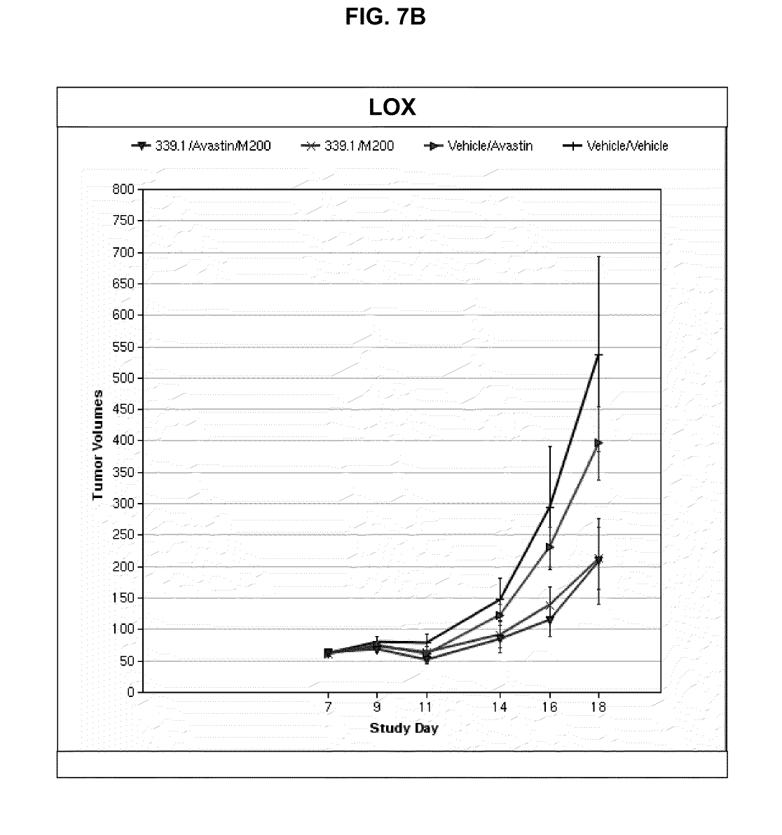

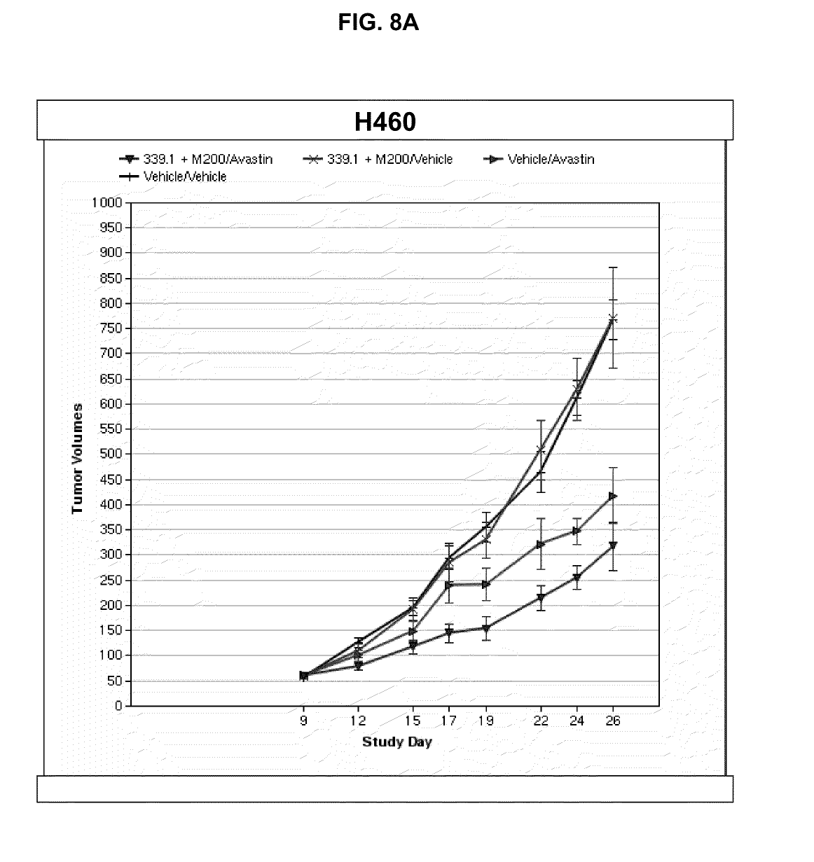

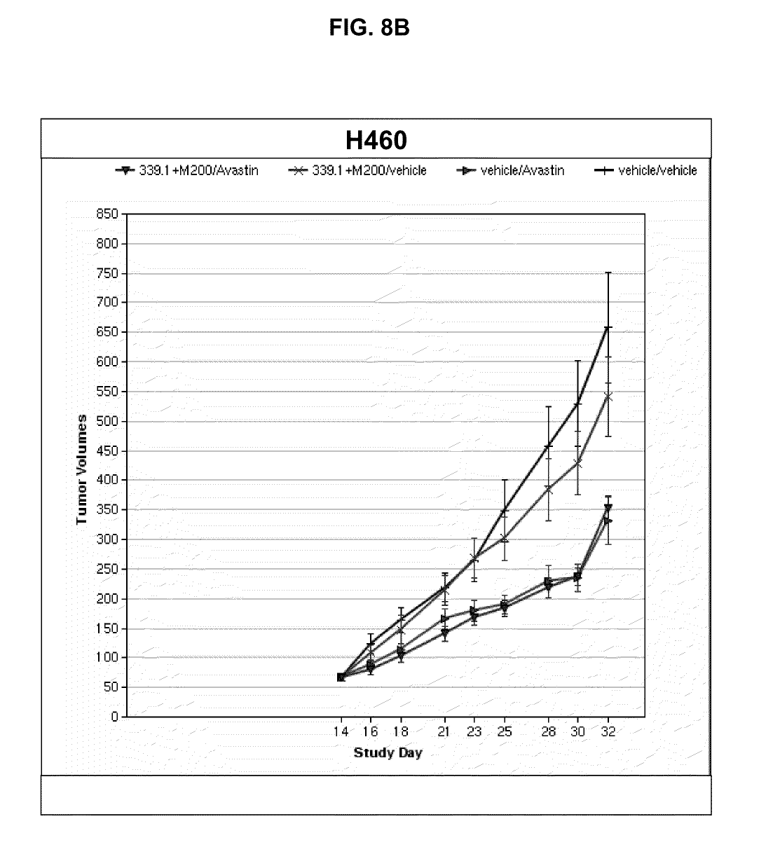

The results of the experiments are illustrated by the plots of tumor volume versus study day shown in FIGS. 6, 7 and 8, respectively. These results suggest a trend for greater tumor reduction when an anti-α5β1 antagonist is used with AVASTIN®.

These results with 339.1 in mouse xenograft models suggest the pharmaceutical combination of volociximab and bevacizumab as a possible treatment in human cancer patients, especially for indications of renal cancer, lung cancer and melanoma.

### Example 5

### Clinical Trial Model for a Pharmaceutical Combination

This example illustrates a possible human clinical trial that may be performed to support the combination of volociximab and SUTENT® for use in treating cancer.

Patients with metastatic renal cell carcinoma are administered SUTENT® orally (50 mg qd, 4 weeks on, 2 weeks off) in combination with intravenous placebo, or SUTENT® orally (50 mg qd, 4 weeks on, 2 weeks off) in combination with intravenous volociximab (either 10 or 15 mg/kg q2w) until disease progression. After treatment, the patients are evaluated to determine the time to disease progression. Patients also are evaluated to determine the duration of response of patients treated with volociximab plus SUTENT® compared to patients treated with placebo plus SUTENT®. Safety and efficacy measurements also are performed, such as by disease-directed radiographic imaging, physical examination, vital sign measurements and other standard laboratory measurements.

Those of skill in the art readily appreciate that the present invention is well adapted to carry out the objects and obtain the ends and advantages mentioned, as well as those inherent therein. The compositions and methods described herein are representative, exemplary embodiments, and are not intended as limitations on the scope of the invention.

While particular embodiments of the present invention have been illustrated and described, it will be apparent to those skilled in the art that various other changes and modifications can be made without departing from the spirit and scope of the invention. It is therefore intended to cover in the appended claims all such changes and modifications that are within the scope of this invention.

The invention illustratively described herein suitably may be practiced in the absence of any element(s) or limitation(s) which is not specifically disclosed herein. The terms and expressions which have been employed are used as terms of description and not of limitation, and there is no intention that in the use of such terms and expressions of excluding any equivalents of the features shown and described or portions thereof.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

All patents and publications are herein incorporated by reference to the same extent as if each individual publication was specifically and individually indicated to be incorporated by reference.

We claim:

1. A pharmaceutical combination for the treatment of cancer comprising an α5β1 antagonist and a tyrosine kinase inhibitor.

2. The combination of claim 1, wherein the cancer is selected from the group consisting of renal cell carcinoma, melanoma, pancreatic cancer, gastrointestinal stromal tumor, bladder cancer, breast cancer, colon cancer, fibrosarcoma, lung cancer, metastatic melanoma, prostate cancer, ovarian cancer, and spleen cancer.

3. The combination of claim 1, wherein the α5β1 antagonist is an antibody or an antigen binding fragment thereof.

4. The combination of claim 3, wherein the antibody comprises a heavy chain comprising SEQ ID NO: 2 and a light chain comprising SEQ ID NO: 4.

5. The combination of claim 3, wherein the antibody comprises a heavy chain variable region comprising SEQ ID NO: 5, a light chain variable region comprising SEQ ID NO: 6, and a human constant region.

6. The combination of claim 1, wherein the tyrosine kinase inhibitor is selected from the group consisting of bis-monocylic, bicyclic and heterocyclic aryl compounds, vinyleneazaindole derivatives, 1-cyclopropyl-4-pyridylquinolones, styryl compounds, styryl-substituted pyridyl compounds, quinazoline derivatives, selenaindoles and selenides, tricyclic polyhydroxylic compounds, benzylphosphonic acid compounds, pyrrole substituted 2-indolinones, aryl urea compounds, sorafenib, sorafenib tosylate, dasatinib, erlotinib, gefitinib, imatinib, lapatinib, nilotinib, sunitinib, sunitinib malate, vandetanib, and bevacizumab.

7. The combination of claim 6, wherein the pyrrole substituted 2-indolinone is sunitinib, or a pharmaceutically acceptable salt thereof.

8. The combination of claim 7, wherein the pyrrole substituted 2-indolinone is sunitinib malate.

9. The combination of claim 8, wherein the combination comprises volociximab and sunitinib malate.

10. The combination of claim 6, wherein the combination comprises volociximab and sorafenib tosylate.

11. The combination of claim 6, wherein the combination comprises volociximab and bevacizumab.

12. The combination of claim 1, wherein the α5β1 antagonist is volociximab formulated for intravenous administration.

13. The combination of claim 12, wherein the volociximab formulation comprises between 1.0 mg/mL and 15.0 mg/mL volociximab, 22 mM to 28 mM citrate, 135 mM to 165 mM sodium chloride, 0.04%-0.06% polysorbate (TWEEN®) 80, at a pH of 5.5 to 7.5.

14. The combination of claim 13, wherein the solution comprises 10.0 mg/mL volociximab, 25 mM citrate, 150 mM sodium chloride, 0.05% polysorbate (TWEEN®) 80, at a pH of 6.5.

15. The combination of claim 8, wherein the tyrosine kinase inhibitor is sunitinib malate formulated as a capsule.

16. The combination of claim 6, wherein the tyrosine kinase inhibitor is sorafenib tosylate formulated as a tablet.

17. The combination of claim 6, wherein the the tyrosine kinase inhibitor is bevacizumab formulated for intravenous administration.

18. A method for treating or preventing cancer comprising administering the pharmaceutical combination of claim 9.

19. A method for treating or preventing cancer comprising administering the pharmaceutical combination of claim 10.

20. A method for treating or preventing cancer comprising administering the pharmaceutical combination of claim 11.


SEQUENCE LISTING (23 sequences)

SEQ ID NO: 1 - DNA, 1353 bp, Artificial
  volociximab complete heavy chain DNA sequence
caggtgcagctgaaggagtcaggacctggcctggtggcgccctcacagagcctgtccatcacatgcaccatctcagggttctcattaaccgactatggtgttcactgggttcgccagcctccaggaaagggtctggagtggctggtagtgatttggagtgatggaagctcaacctataattcagctctcaaatccagaatgaccatcaggaaggacaactccaagagccaagttttcttaataatgaacagtctccaaactgatgactcagccatgtactactgtgccagacatggaacttactacggaatgactacgacgggggatgctttggactactggggtcaaggaacctcagtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 2 - protein, 451 aa, Artificial
  volociximab complete heavy chain amino acid sequence
QVQLKESGPGLVAPSQSLSITCTISGFSLTDYGVHWVRQPPGKGLEWLVVIWSDGSSTYNSALKSRMTIRKDNSKSQVFLIMNSLQTDDSAMYYCARHGTYYGMTTTGDALDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - DNA, 645 bp, Artificial
  volociximab complete light chain DNA sequence
caaattgttctcacccagtctccagcaatcatgtctgcatctctaggggaacgggtcaccatgacctgcactgccagttcaagtgtaagttccaattacttgcactggtaccagcagaagccaggatccgcccccaatctctggatttatagcacatccaacctggcttctggagtcccagctcgtttcagtggcagtgggtctgggacctcttactctctcacaatcagcagcatggaggctgaagatgctgccacttattactgccaccagtatcttcgttccccaccgacgttcggtggaggcaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 4 - protein, 215 aa, Artificial
  volociximab complete light chain amino acid sequence
QIVLTQSPAIMSASLGERVTMTCTASSSVSSNYLHWYQQKPGSAPNLWIYSTSNLASGVPARFSGSGSGTSYSLTISSMEAEDAATYYCHQYLRSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 124 aa, Mus musculus
QVQLKESGPGLVAPSQSLSITCTISGFSLTDYGVHWVRQPPGKGLEWLVVIWSDGSSTYNSALKSRMTIRKDNSKSQVFLIMNSLQTDDSAMYYCARHGTYYGMTTTGDALDYWGQGTSVTVSS

SEQ ID NO: 6 - protein, 109 aa, Mus musculus
QIVLTQSPAIMSASLGERVTMTCTASSSVSSNYLHWYQQKPGSAPNLWIYSTSNLASGVPARFSGSGSGTSYSLTISSMEAEDAATYYCHQYLRSPPTFGGGTKLEIKR

SEQ ID NO: 7 - DNA, 696 bp, Artificial
  F200 complete heavy chain DNA sequence
caggtgcagctgaaggagtcaggacctggcctggtggcgccctcacagagcctgtccatcacatgcaccatctcagggttctcattaaccgactatggtgttcactgggttcgccagcctccaggaaagggtctggagtggctggtagtgatttggagtgatggaagctcaacctataattcagctctcaaatccagaatgaccatcaggaaggacaactccaagagccaagttttcttaataatgaacagtctccaaactgatgactcagccatgtactactgtgccagacatggaacttactacggaatgactacgacgggggatgctttggactactggggtcaaggaacctcagtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatca

SEQ ID NO: 8 - protein, 232 aa, Artificial
  F200 complete heavy chain amino acid sequence
QVQLKESGPGLVAPSQSLSITCTISGFSLTDYGVHWVRQPPGKGLEWLVVIWSDGSSTYNSALKSRMTIRKDNSKSQVFLIMNSLQTDDSAMYYCARHGTYYGMTTTGDALDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPS

SEQ ID NO: 9 - protein, 124 aa, Artificial
  amino acid sequence of heavy chain variable region for humanized antibody #1
QVQLVESGPGLVQPGGSLRISCAISGFSLTDYGVHWVRQAPGKGLEWLVVIWSDGSSTYNSALKSRMTISKDNSKSTVYLQMNSLRAEDTAMYYCARHGTYYGMTTTGDALDYWGQGTLVTVSS

SEQ ID NO: 10 - protein, 109 aa, Artificial
  amino acid sequence of light chain variable region for humanized antibody #1
DIQLTQSPSSMSASLGDRVTMTCTASSSVSSNYLHWYQQKPGKAPNLWIYSTSNLASGVPSRFSGSGSGTDYTLTISSMQPEDFATYYCHQYLRSPPTFGQGTKLEIKR

SEQ ID NO: 11 - protein, 124 aa, Artificial
  amino acid sequence of heavy chain variable region for humanized antibody #2
EVQLVESGGGLVQPGGSLRISCAISGFSLTDYGVHWVRQAPGKGLEWLVVIWSDGSSTYNSALKSRMTISKDNSKNTVYLQMNSLRAEDTAVYYCARHGTYYGMTTTGDALDYWGQGTLVTVSS

SEQ ID NO: 12 - protein, 109 aa, Artificial
  amino acid sequence of light chain variable region for humanized antibody #2
DIQLTQSPSSLSASVGDRVTMTCTASSSVSSNYLHWYQQKPGKAPKLWIYSTSNLASGVPSRFSGSGSGTDYTLTISSMQPEDFATYYCHQYLRSPPTFGQGTKLEIKR

SEQ ID NO: 13 - protein, 124 aa, Artificial
  amino acid sequence of heavy chain variable region for humanized antibody #3
EVQLVESGGGLVQPGGSLRLSCAASGFSLTDYGVHWVRQAPGKGLEWVSVIWSDGSSTYNSALKSRFTISRDNSKNTLYLQMNSLRAEDTAVYYCARHGTYYGMTTTGDALDYWGQGTLVTVSS

SEQ ID NO: 14 - protein, 109 aa, Artificial
  amino acid sequence of light chain variable region for humanized antibody #3
DIQMTQSPSSLSASVGDRVTITCTASSSVSSNYLHWYQQKPGKAPKLLIYSTSNLASGVPSRFSGSGSGTDFTLTISSLQPEDFATYYCHQYLRSPPTFGQGTKVEIKR

SEQ ID NO: 15 - protein, 124 aa, Artificial
  amino acid sequence of heavy chain variable region for humanized antibody #4
EVQLVESGGGLVQPGGSLRLSCAISGFSLTDYGVHWVRQAPGKGLEWLVVIWSDGSSTYNSALKSRMTISKDNSKSTVYLQMNSLRAEDTAVYYCARHGTYYGMTTTGDALDYWGQGTLVTVSS

SEQ ID NO: 16 - protein, 109 aa, Artificial
  amino acid sequence of light chain variable region for humanized antibody #4
DIQLTQSPSSLSASVGDRVTITCTASSSVSSNYLHWYQQKPGKAPKLWIYSTSNLASGVPSRFSGSGSGTDYTLTISSLQPEDFATYYCHQYLRSPPTFGQGTKVEIKR

SEQ ID NO: 17 - protein, 124 aa, Artificial
  amino acid sequence of heavy chain variable region for humanized antibody #5
QVQLVESGGGLVQPGGSLRISCAISGFSLTDYGVHWVRQAPGKGLEWLVVIWSDGSSTYNSALKSRMTISKDNSKSTVYLQMNSLRAEDTAMYYCARHGTYYGMTTTGDALDYWGQGTLVTVSS

SEQ ID NO: 18 - protein, 109 aa, Artificial
  amino acid sequence of light chain variable region for humanized antibody #5
DIQLTQSPSSLSASVGDRVTMTCTASSSVSSNYLHWYQQKPGKAPKLWIYSTSNLASGVPSRFSGSGSGTDYTLTISSLQPEDFATYYCHQYLRSPPTFGQGTKVEIKR

SEQ ID NO: 19 - protein, 451 aa, Artificial
  amino acid sequence of complete heavy chain for humanizedantibody #6
EVQLVESGGGLVQPGGSLRLSCAASGFSLTDYGVHWVRQAPGKGLEWLVVIWSDGSSTYNSALKSRMTISKDNAKNTVYLQMNSLRAEDTAVYYCARHGTYYGMTTTGDALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 215 aa, Artificial
  amino acid sequence of complete light chain for humanizedantibody #6
EIVLTQSPATLSLSPGERATLSCTASSSVSSNYLHWYQQKPGQAPRLLIYSTSNLASGVPARFSGSGSGTSYTLTISSLEPEDFAVYYCHQYLRSPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 9 aa, Artificial
  alpha 5 beta 1 antagonist peptide
CRRETAWAC

SEQ ID NO: 22 - protein, 132 aa, Artificial
  heavy chain variable region for mouse volociximab analog
MDIRLSLAFLVLFIKGVQCEVYLVESGGGSVQPGRSTKLSCAASEFTFSTSSMAWVRQAPTKGLEWVATISSSGEYTYYRDSVKGRFTISRDNAKSTLYLQMNSLRSEDTATYYCTRGVLYWGQGVMVTVSS

SEQ ID NO: 23 - protein, 125 aa, Artificial
  light chain variable region for mouse volociximab analog
MAPVQLLGLLLLCLPAMRCDIQMTQSPSLLSASVGDRVTLNCKSSQNIYKNLNWYQQKLGEPPKLLIHYTDNLQAGIPSRFSGSGSGTDYTLTISSLHPDDVATYFCFQYNRGPTFGAGTKLELK